(19) United States
(12) Patent Application Publication
(10) Pub. No.: US 2022/0016289 A1
(43) Pub. Date: Jan. 20, 2022

(54) CENTRALIZED PROGRAMMABLE COMPUTER CONTROLLED AUTOMATED BIOCIDAL/DISINFECTION/CHEMICAL DISTRIBUTION SYSTEM

(71) Applicant: SYMBIOS, LLC, Deerfield Beach, FL (US)

(72) Inventor: Ronnie N. Collins, Boca Raton, FL (US)

(21) Appl. No.: 17/375,816

(22) Filed: Jul. 14, 2021

Related U.S. Application Data
(60) Provisional application No. 63/051,691, filed on Jul. 14, 2020.

(51) Int. Cl. A61L 2/24; A61L 2/22; A61L 9/14

(52) U.S. Cl. CPC A61L 2/24; A61L 2/22; A61L 9/14; A61L 2209/134; A61L 2202/25; A61L 2209/111; A61L 2202/14

(57) ABSTRACT

A disinfection system includes a controller, disinfectant bank, valve manifold, and fluid distribution network that is terminated by nozzles in each of a plurality of spaces. The controller is operable to control pumping of disinfectant to various groups of nozzles in each of the spaces in order to disinfect the spaces at scheduled or unscheduled times. The nozzles are remotely actuated by wireless network communications from the controller, and the controller is responsive to sensors in each of the spaces that can indicate the presence of a person in the spaces. When a person is in a space, disinfection is inhibited in that space.

### CROSS REFERENCE

This application claims priority to U.S. Provisional Application No. 63/051,691, filed Jul. 14, 2020, the entirety of which is hereby incorporated by reference.

### FIELD OF THE DISCLOSURE

The present invention relates generally to systems, methods, and apparatuses for disinfecting living spaces, and, more particularly, relates to a centralized automated.

### BACKGROUND OF THE DISCLOSURE

With the ongoing pandemic caused by the spread of the SARS-CoV-2 virus, it has become apparent that frequent disinfection is useful in preventing spread of viruses as well as infection. Currently the most common methods of disinfecting spaces that people use include, for example, portable sprayer/fogging systems that distribute disinfectant via aerosolization of chemicals. This is a manual, and therefore labor intensive process. Presently, this kind of process is used for all kinds of spaces, including airplanes, rental cars, buildings, and so on, and represents a substantial cost in mitigating the spread of infection.

Therefore, a need exists to overcome the problems with the prior art as discussed above.

### SUMMARY OF THE INVENTION

In accordance with some embodiments of the inventive disclosure, there is provided an automated disinfection system that includes a controller, a pump that is coupled to and operated by the controller, a reservoir including at least one disinfectant cartridge that contains a disinfectant agent, the reservoir being fluidly coupled to the pump, and a fluid distribution network. The fluid distribution network includes a plurality of conduits, each conduit being routed to a different one of a plurality of spaces and separately connected to the pump by a respective one of a plurality of controllable valves at a pump end of each one of the plurality of conduits. Each one of the plurality of conduits is branched, at a terminal end, to a plurality of remotely controlled nozzles, and each one of the plurality of nozzles is disposed in one of the plurality of spaces. The controller is operable to, according to a schedule, operate the pump, the plurality of controllable valves, and remotely controlled nozzles to sequentially distribute disinfectant from the disinfectant bank to each one of the plurality of spaces.

In accordance with a further feature, the system further includes, in at least one of the plurality of spaces, at least one biosensor that is operably coupled to the controller, wherein the biosensor indicates to the controller whether a person is present in the at least one of the plurality of spaces, and wherein upon detecting a person in the at least one of the plurality of spaces the controller inhibits administering the disinfectant in the at least one of the plurality of spaces in which the person was detected.

In accordance with a further feature, the system further includes, in at least one of the plurality of spaces, an access control that is operably coupled to the controller, and which is operated by the controller to prevent access to the at least one of the plurality of spaces upon commencement of administration of the disinfectant into the at least one of the plurality of spaces.

In accordance with a further feature, the plurality of nozzles are each remotely controlled using wireless network communications, and wherein at least of the plurality of nozzles are controlled by the controller as a group with one of the plurality of spaces.

In accordance with a further feature, the controller and pump are housed together on a mobile platform.

In accordance with a further feature, the controller and pump are housed together on a fixed platform in a structure.

In accordance with a further feature, the controller includes a wireless network communication transceiver, and wherein the schedule is received from a remote server via the wireless network communication transceiver.

In accordance with some embodiments of the inventive disclosure, there is provided an automated disinfection system that includes a controller including instruction code stored in a non-volatile computer readable medium that is instantiated by the controller for execution by the controller, a pump that is coupled to and operated by the controller, a reservoir including at least one disinfectant cartridge that contains a disinfectant agent, the reservoir being fluidly coupled to the pump, and a valve manifold coupled to the pump at an intake and including a plurality of controllable valves. The system further includes a fluid distribution network including a plurality of conduits, with each conduit being routed to a different one of a plurality of spaces and separately connected to the pump by a respective one of the plurality of controllable valves at a pump end of each one of the plurality of conduits. Each one of the plurality of conduits further being branched, at a terminal end, to a plurality of remotely controlled nozzles, and each one of the plurality of nozzles is disposed in one of the plurality of spaces. The system further includes a plurality of sensors distributed among the plurality of spaces, where each one of the plurality of spaces include at least one sensor, and each sensor of the plurality of sensors being coupled to the controller and operable to indicate to the controller a signal indicating a presence of a person in the respective space. Wherein the controller is operable to, according to a schedule, operate the pump, the plurality of controllable valves, and remotely controlled nozzles to sequentially distribute disinfectant from the disinfectant bank to each one of the plurality of spaces, and to further inhibit a scheduled disinfection process for a given one of the plurality of spaces when the respective sensor in the given one of the plurality of spaces indicates a person is in the given one of the plurality of spaces.

In accordance with a further feature, the at least one sensor includes a thermal imaging sensor capable of identifying a person, a temperature of the person, and wherein the controller is configured to determine whether the temperature of the person is above an preselected threshold, and to commence an unscheduled disinfection of the space in which the person is located.

In accordance with a further feature, the system further includes, in at least one of the plurality of spaces, an access control that is operably coupled to the controller, and which is operated by the controller to prevent access to the at least one of the plurality of spaces upon commencement of administration of the disinfectant into the at least one of the plurality of spaces.

In accordance with a further feature, the plurality of nozzles are each remotely controlled using wireless network communications, and wherein at least of the plurality of nozzles are controlled by the controller as a group with one of the plurality of spaces.

In accordance with a further feature, the controller and pump are housed together on a mobile platform.

In accordance with a further feature, the controller and pump are housed together on a fixed platform in a structure.

In accordance with a further feature, the controller includes a wireless network communication transceiver, and wherein the schedule is received from a remote server via the wireless network communication transceiver.

In accordance with some embodiments of the inventive disclosure, there is provided a method for disinfecting a plurality of spaces, the method including, at a controller, determining that a disinfection process is to commence for at least one of a plurality of spaces. The method further including, responsive to determining that the disinfection process is to commence, the controller receiving a signal from a sensor in the at least one of the plurality of spaces. When the signal indicates that the least one of the plurality of spaces does not contain any persons, the controller activates one of a plurality of valves of a valve manifold and opening the valve, transmits a signal that causes at least one nozzle in the space to activate, and activates a pump that is coupled to a disinfectant bank on a first side of the pump and to the valve manifold on a second side of the pump to commence pumping disinfectant into the valve manifold. Wherein the disinfectant is pumped through valve manifold into a respective one of a plurality of conduits that is connected at a pump end to the one of the plurality of valves in the valve manifold and at a terminal end to the at least one nozzle where the at least one nozzle distributes aerosolized disinfectant into the space.

Although the invention is illustrated and described herein as embodied in a centralized automated disinfection system, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one as or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

“In the description of the embodiments of the present invention, unless otherwise specified, azimuth or positional relationships indicated by terms such as “up”, “down”, “left”, “right”, “inside”, “outside”, “front”, “back”, “head”, “tail” and so on, are azimuth or positional relationships based on the drawings, which are only to facilitate description of the embodiments of the present invention and simplify the description, but not to indicate or imply that the devices or components must have a specific azimuth, or be constructed or operated in the specific azimuth, which thus cannot be understood as a limitation to the embodiments of the present invention. Furthermore, terms such as “first”, “second”, “third” and so on are only used for descriptive purposes, and cannot be construed as indicating or implying relative importance.

In the description of the embodiments of the present invention, it should be noted that, unless otherwise clearly defined and limited, terms such as “installed”, “coupled”, “connected” should be broadly interpreted, for example, it may be fixedly connected, or may be detachably connected, or integrally connected; it may be mechanically connected, or may be electrically connected; it may be directly connected, or may be indirectly connected via an intermediate medium. As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. To the extent that the inventive disclosure relies on or uses software or computer implemented embodiments, the terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system. Those skilled in the art can understand the specific meanings of the above-mentioned terms in the embodiments of the present invention according to the specific circumstances.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a block schematic diagram of a centralized automated disinfection system, in accordance with some embodiments;

FIG. 2 is a block schematic diagram of the system of FIG. 1 extended to multiple spaces, in accordance with some embodiments;

FIG. 3 is a block schematic diagram of a nozzle assembly, in accordance with some embodiments;

FIGS. 4A & 4B show various arrangements for grouping nozzle assemblies, to deliver a disinfectant into a space, in accordance with some embodiments;

FIGS. 5A and 5B show sequential disinfection of different spaces, in accordance with some embodiments;

FIG. 6 shows a representation of a schedule data structure for use with a disinfection system, in accordance with some embodiments;

FIG. 7 shows a flow chart diagram of a method of operating a disinfection system, in accordance with some embodiments; and

FIG. 8 shows an interface diagram of sequential interface presentations to a user for editing, creating a disinfection schedule or running an unscheduled disinfection operation, in accordance with some embodiments.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

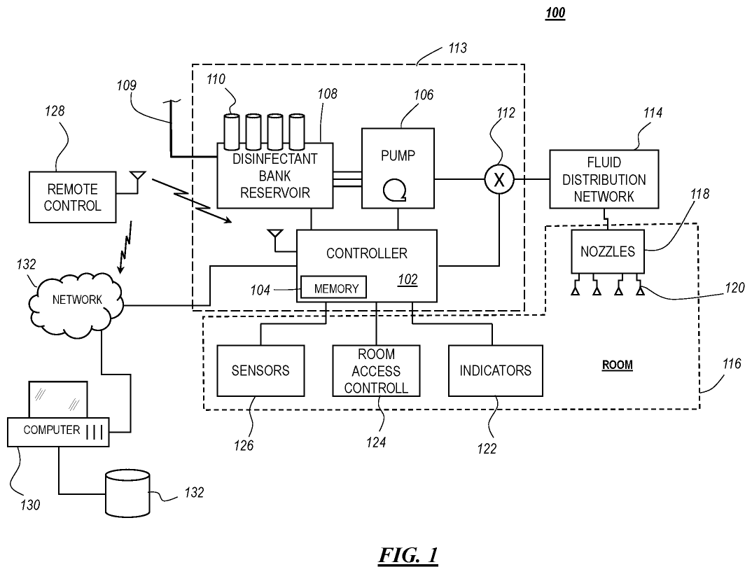

FIG. 1 is a block schematic diagram of a centralized automated disinfection system 100, in accordance with some embodiments. The system 100 includes a controller 102 that is a computer operated controller or central processing unit (CPU) which includes a microprocessor and memory 104. The memory 104 is an aggregate memory representing both volatile and non-volatile forms of memory, including random access memory (RANI) for both instantiating instruction code and scratch pad memory, as well as long term storage for schedules, and other data. The controller 102 operates a pump system 106 and a valve system 112 that can be provided between the pump system and a fluid distribution network 114. Further, the controller 102 can be operably coupled to a disinfectant bank or reservoir 108 in which there can be one or more supply containers 110 of disinfecting agents. There can be different types of disinfecting agents as well. It is contemplated that the disinfectant containers 110 can contain a pre-diluted mixture, or that a water source 109 can be connected to the disinfectant bank 108 to allow mixing of water with disinfectant agents in the containers 110 for distribution. The controller 102 can be operably coupled to the disinfectant bank 108 to detect the levels of disinfectant agents in the supply containers in order to prompt an administrator to replace the containers when the supply runs lows.

The pump 106, under control of the controller 102, pumps material from the disinfectant bank 108 into the fluid distribution network 114 through selected valves 112. The term “fluid” as used here can refer to both wet (liquid) and dry (gaseous/particulate) fluids as well as aerosolized droplets of fluid suspending in air. The fluid distribution network 114 carries the fluids to one or more nozzles 118 in a room 116 or space being disinfected. Generally, the nozzles 118 are fixed, being mounted in the space. The nozzles 118 are shown together here, but in practice are spaced throughout the room 116 to ensure adequate coverage in the room 116 of the disinfectant material being administered. In some embodiments the nozzles 118 can include electrostatic nozzles. Further, the room 116 can contain one or more sensors 126 that are also operably coupled to the controller 102 to detect people who may be in the room 116. For example, motion sensors and/or thermal sensors, as well as optical recognition (cameras) can be used to ensure that the room 116 is free of people before activating the pump 106 and opening the valve 112. Further, the sensors 126 can be used to shut down the disinfection process if a person is subsequently detected during the disinfection process. In some embodiments the controller 102 can be coupled to a room access control mechanism 124, such as a door lock, in order to prevent ingress into the room 116 during the disinfection process. Additionally, an indicator 122 can be controlled by the controller 102 to give visual and/or audible indication to people outside of the room 116 that a disinfection process is underway, or whether it is clear to enter the room 116.

The controller 102 can include both wireless and wired network interfaces. A wireless interface can be used to receive commands from a remote control device 128, which can be, for example, portable computing device running an application program designed to operate and interface with the controller 102. In some embodiments the controller 102 can transmit data to the remote control unit 128. In some embodiments the remote control unit 128 is less sophisticated, and merely provides command codes to the controller 102, such as a shutdown command, start command, etc. The controller 102 can also be connected to a network 132, which can be local area network or a wide area network (e.g. the Internet) that allows people to remotely access the controller 102 to input information such as operation schedules, as well as to see data maintained by the controller 102, such as event logs, the status of disinfectant levels in the disinfectant banks, and so on.

In operation, the controller 102 can be programmed with a schedule of operation, which can follow, for example, a class schedule in a school, a route schedule in a vehicle, or other schedules as may be contemplated by those skilled in the art. The system 100 can also be manually activated if necessary, meaning activated outside of a scheduled time. The controller 102 includes, or is coupled to a clock, or other event indicator (e.g. location, status of the room), and upon the occurrence of a predefined event, such as a time, or arriving at a location, or the room 116 emptying of people, the controller 102 commences a disinfection process in which the valve 112 is opened and the pump 106 is activated to cause disinfectant materials to be delivered to the nozzles and distributed into the room. In some embodiments the nozzles 118 may also be controllable to turn on/off or to configure a type of operation/spray pattern. At different times, or upon different events, different amounts or kinds of disinfecting materials can be used/distributed into the room 116. For example, in a school, between classes a mild disinfectant can be used, and at night after the school is closed a more powerful disinfectant can be used, or applied for a longer duration, and allowed to dissipate before the school is opened again.

In some embodiments, the controller 102, pump 106, disinfectant bank 108, and valve 112 can be mounted together in a portable or mobile unit, allowing the unit 113 to be moved from one location to another as needed. The fluid network 114 contains a plurality of fluid conduits or lines that route fluid to the nozzles 118. Different ones of the conduits can connect to different groups of nozzles 118. The disinfectant bank/reservoir 108 can control which disinfectants 110 are used for a particular disinfection operation, including mixing of different types of disinfectants for simultaneous application. As mentioned, the fluid network 114 can contain a plurality of conduits, with each one of the conduits having a connector for coupling to a corresponding coupling of the valve 112. Thus, it is contemplated that the valve 112 can be a valve bank with separately controllable valves that are operated by the controller 102.

It is contemplated that in some embodiments the system 100 or substantial portions of the system 100 can be integrated, or operative with an existing heating ventilation and air conditioning (HVAC) system. The conduits of the fluid delivery system, for example, can be routed through exiting ducts, and the nozzles can be located adjacent the ducts, as well as on other ceiling locations. In some embodiments the controller 102 can be coupled to the HVAC system and is operative to control at least some operation of the HVAC system. For example, one of the sensors 126 can be a humidistat of the HVAC system. Upon application of a disinfectant the humidity in a space can be elevated, causing the HVAC system to undertake a dehumidification process to remove moisture from the air. However, this may result in the disinfectant being removed too soon. Thus, the controller 102 can be configured to inhibit dehumidification for a time after application of a disinfectant. Thereafter the HVAC system can commence a dehumidification process to restore the humidity in the space to a preferred level.

A remote computer 130, such as, for example, a server or data center, can be accessed by users, such as by using the remote control device 128. In some embodiments, the remote control device 128 can be a smartphone device running an application program for communicating with the controller 102 and/or the remote computer 130. The remote computer 130 can be coupled to a data store 132 in which data such as schedules can be stored for a given system 100. Thus, in some embodiments, a user can use their remote control device 128 to access the remote computer 130, and build, change schedules, define groups of nozzles in spaces, and so on. This information can be relayed to the controller 102 for performance of the schedule by the controller 102 in the system 100. It should be apparent to those skilled in the art that room 116 can represent one of many rooms or spaces being disinfected by the system 100. Each room can be disinfected in turn according to a schedule.

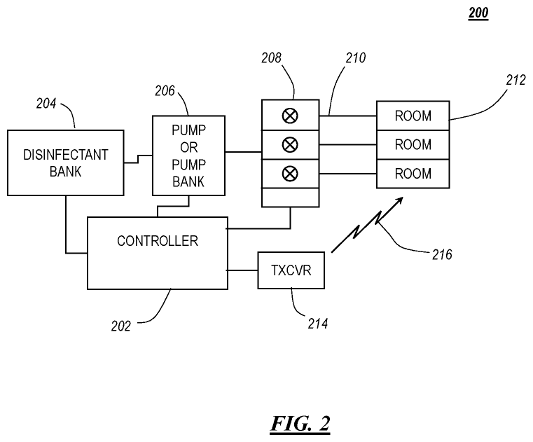

FIG. 2 is a block schematic diagram of the system 100 of FIG. 1 extended to multiple spaces. This figure shows that the controller 202 (equivalent to 102) can operate in multiple rooms 212, with each room (equivalent to 116) having a dedicated valve manifold 208 and/or a dedicated pump 206 to provide disinfectant materials from the disinfectant bank 204 to the rooms 212, to be dispersed in the room by nozzles (e.g. 118) mounted in each room 212. Each room can have its own schedule that is administered by the controller 202, and which can be input into the controller 202 by a user using the remote computer (e.g. 130) or remote control device (e.g. 128). The rooms, can be, for example, school rooms, hotel rooms, offices, etc. Upon the scheduled time occurring for a given room 212 for a disinfection operation the controller 202 opens the corresponding valve of the valve manifold 208 which is connected to a corresponding conduit 210 and activates the pump 206 to commence pumping disinfectant through the opened valve 208 and the corresponding conduit 210 to nozzles in the room 212 being disinfected. Further, the controller 202 can control the disinfectant bank 204 to activate particular ones of different types of disinfectant to be combined and distributed into the room 212, or a single type of disinfectant can be selected. It is also contemplated that the nozzles in the rooms 212 can be remotely operated or remotely actuated using wireless communications. Thus, a transceiver 214 operably connected to the controller 202 can be used to transmit wireless signals to selected ones of the nozzles to activate them to achieve a preferred or selected type of dispersion for the given disinfectant or combination of disinfectants being used. The transceiver 214 can operate using known wireless networking communications interfaces such as, for example, Wi-Fi, Bluetooth, mesh networking, or other equivalent wireless network communications. It is further contemplated, as will be shown, that various different nozzle types can be provided in the rooms 212 and selectively grouped and controlled together by nozzle type using the communications and wireless signals 216 transmitted by the wireless transceiver 214. That is different types of disinfectant may require different nozzle types to achieve proper dispersion within the rooms.

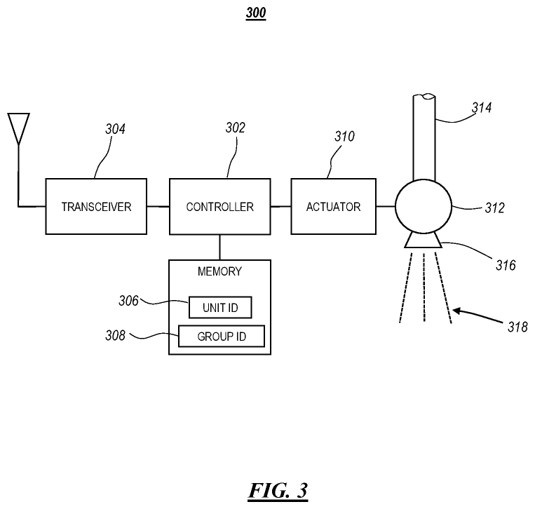

FIG. 3 is a block schematic diagram of a nozzle assembly 300, in accordance with some embodiments. The nozzle assembly 300 includes its own controller 302, which can be a microprocessor, the control operation of the nozzle assembly. The controller 302 is interfaced with a wireless network communications transceiver 304 which can receive and transmit wireless signals with a system controller (e.g. 102). The controller 302 is further interfaced with a memory in which there can be stored a unit ID 306 that identifies the particular nozzle assembly 300, and a group ID 308 that identifies a group of nozzles to which the nozzle assembly 300 belongs or is assigned to for operation. The nozzle assembly 300 can be included in multiple groups to achieve various types of disinfectant operations. The controller 302 is further interfaced with an actuator 310 that controls a nozzle valve 312. The nozzle valve 312 is operable to open or close, as well as open to various degrees to control the amount of disinfectant passing through the nozzle over a given time (e.g. rate). The nozzle valve provides disinfectant through a nozzle horn 316 that controls a distribution pattern of the dispersed disinfectant 318. The nozzle valve 310 is fed by a conduit 314 that is part of the fluid distribution network (e.g. 114). The nozzle valve 312 and nozzle horn 316 can take any of a number of known nozzle configurations including, for example, electrostatic nozzles, misting nozzles, fogging nozzles, to name a few types of nozzles.

To activate the nozzle assembly 300 system controller transmits a signal that is received by transceiver 304. The signal can include either the unit ID 306 or the group ID 308. Upon receiving either the unit ID 306 or the group ID 308 the controller will operate the actuator 3102 enable the nozzle valve 312. Disinfectant provided to the nozzle valve 312 through the conduit 314 by a pump (e.g. 106) under pressure passes through the nozzle horn 316 and is dispersed in the intended pattern in the room. Accordingly when the controller of the system wants to actuate a group of nozzles located, for example, together in a room, the system controller will broadcast the group ID 308 along with an indication to enable the nozzles which can be in, for example, a bit field transmitted by the system controller. That is, communications can be defined such that a digital word or words of certain preselected sizes can include a group ID, a nozzle ID or unit ID, and bits that indicate to open or close the nozzle or nozzles. It is further contemplated that a digital assistant could be used to transmit commands to the system controller, where a voice recognition interface can receive spoken commands and then generate a control signal or digital message that is relayed by the digital assistant to the controller. Examples of a digital assistant include those sold by Amazon, Inc. known as “Alexa,” and by Apple, Inc. as “Siri.”

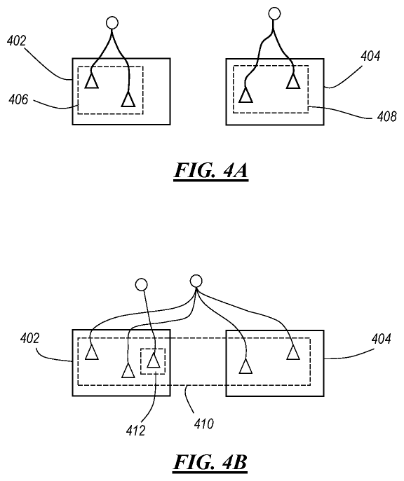

FIGS. 4A & 4B show various arrangements for grouping nozzle assemblies, to deliver a disinfectant into a space, in accordance with some embodiments. In the figures there are shown representations of two rooms including room 402 in room 404. The rooms 402, 404 represent different ones of a plurality of spaces being disinfected by the disinfection system (e.g. 100). In room 402 there is a group 406 of nozzles and in room 404 there is a second group 408 of nozzles. Each of the groups 406, 408 can be coupled to a different conduit of the fluid delivery network. According to a schedule then, group 406 can be activated or opened by transmission of a group ID corresponding to group 406. Each nozzle in group 406 has been provisioned with the same group ID, so that when all of the nozzles in group 406 receive that group identifier they, in unison turn on or turn off, respectively, as indicated in the received transmission. Likewise, all of the nozzles in group 408 have been provisioned with a different group identifier from that used for group 406. Thus, to enable the nozzles in group 408 the controller transmits the group identifier corresponding to group 408. In FIG. 4B, the same two rooms 402, 404 are shown but the nozzles are grouped differently. For example, nozzles in both rooms 402, 404 are included in group 410. Thus with respect to group 401 rooms 402, 404 represent one space even though they can be physically separated. Similarly within room 402 there can be one or more nozzles in a separate group 412. The reason for this is that the nozzles in group 410 and the nozzle or nozzles in group 412 can be different types of nozzles that are each suitable for different types of disinfectant.

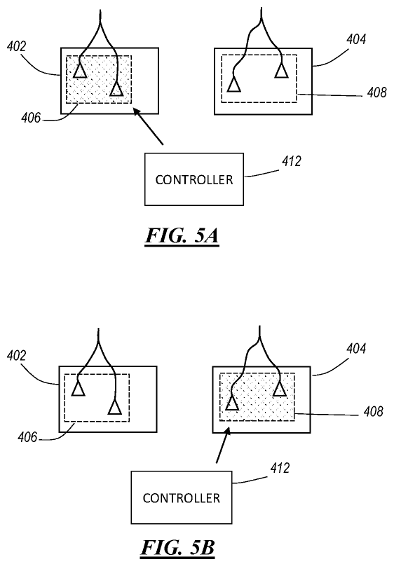

FIGS. 5A and 5B show sequential disinfection of different spaces, in accordance with some embodiments. In FIG. 5A the controller 412 (equivalent to controller 102) has activated the nozzles in group 406 in room 402 and disinfectant is dispersed into room 402 through the nozzles in group 406. The nozzles in group 408 in room 404 have not been activated because it is not there turn yet in the schedule of the present example. Controller 412 is either prompted or has a copy of a disinfecting schedule and upon schedule time to commence disinfection of room 402 the controller 412 transmits the nozzle activation signal by broadcasting the group ID for group 406 along with a bit or bits indicating that the nozzles in group 406 R2 turn on or otherwise activate. At the end of the schedule disinfection time the controller 412 will again transmit the group ID for group 406 along with an indication that the nozzles are to turn off or deactivate. In FIG. 5B, for example, disinfection of room 402 using the nozzles of group 406 has been completed and those nozzles are deactivated. However, according to the schedule, of the present example room 406 is next to be disinfected and accordingly the controller 412 transmits the group ID for group 408 which is received by each one of the nozzles in group 406 which in turn responsive to the activation signal open their respective nozzle valves and disinfectant is pumped through the nozzles of group 408 dispersing disinfectant into the room 404. Thus, according to the present example, the rooms 402, 404 are sequentially disinfected under control of the controller 412 which selectively activates the nozzles in the corresponding groups 406, 408 at their respective schedule disinfection times. This simple example can be expanded to a large number of rooms and the principal remains the same, where groups of nozzles for the different spaces being disinfected are selectively activated or deactivated according to the schedule and disinfectant is provided through the fluid delivery network to the respective nozzles of the groups in their scheduled times. Likewise the respective valves (e.g. 208) at the pump end of the fluid delivery network are selectively activated (opened or closed).

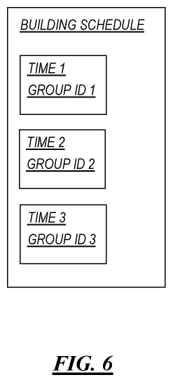

FIG. 6 shows a representation of a schedule data structure for use with a disinfection system, in accordance with some embodiments. The data structure is for a particular building or structure having a plurality of spaces that are to be disinfected according to a schedule. Each one of the plurality of spaces has one or more nozzles which are assigned to a common group for that space. Thus, the schedule can include multiple different group identifiers each of which are scheduled to be activated for a different time. This schedule can be maintained or stored at the controller of the system (e.g. controller 102), as well as at a remote computer or server that is in communication with the controller, in which can be accessed and edited by a user at the server, wherein the server can then forward the schedule to the controller for performance of the corresponding disinfection regimen.

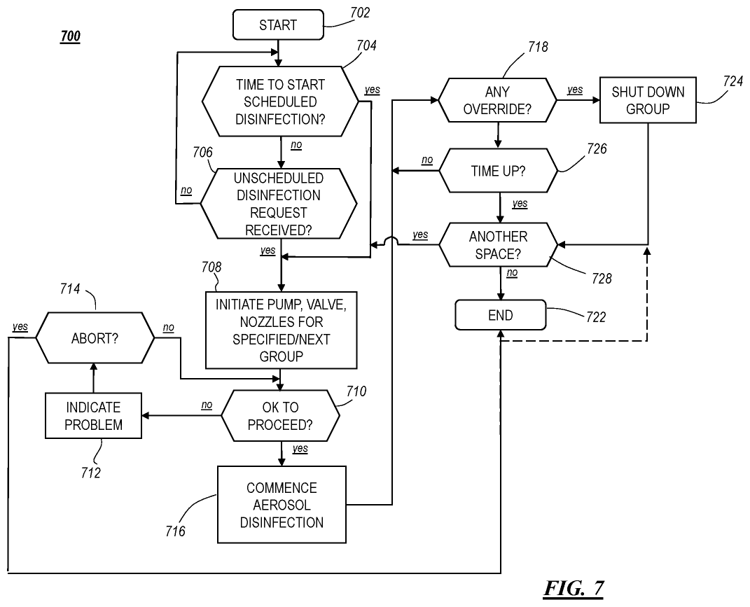

FIG. 7 shows a flow chart diagram of a method 700 of operating a disinfection system, in accordance with some embodiments. At the start 702 the controller unit (e.g. 113) is provided in a building or structure and appropriately coupled to the fluid distribution network installed in the building. Further, the control unit is provided with a disinfection schedule which indicates which nozzle groups to be activated at particular times, which valves are to be opened for the corresponding conduit feeds to those nozzle groups, and which disinfectant agents are to then be pumped into the fluid distribution network conduits to the nozzle groups. In step 706 the method 700 determines whether it is time to start schedule disinfection. If, according to the schedule, it is time to commence a schedule disinfection process, the method 700 skips down to step 708. However, if in step 704 it is not time to start a schedule disinfection the method 700 can determine if there has been an unscheduled disinfection request received in step 706. It is further contemplated that the system can monitor a room or space using, for example, thermal imaging (e.g. one of the sensors 126) to identify persons in the space, and detect whether any person in the space is exhibiting higher than normal temperature, which can indicate that the person is exhibiting a fever, implying the person may be ill and possibly infectious. Accordingly, detection of a person having a higher than normal temperature can cause the system to initiate an unscheduled disinfection of the space once the person has left the space. Thus, an unscheduled disinfection request can arise automatically by the system itself. If it is neither time to start a schedule disinfection and no unscheduled disinfection request has been received the method 700 simply continues to loop through step 704 and 706. In step 708, it is either time for a schedule disinfection or an unscheduled disinfection request has been received, the system (e.g. 100) initiates the pump, selects the appropriate valve, transmits an activation message to the specified nozzle group, and selects the appropriate disinfectant which can be indicated in the schedule data as well. The method 700 then continues to step 710 where sensors corresponding to the space to be disinfected can be checked to determine whether there is anyone in the space. This can include, for example, using thermal sensors, audio sensors, or any other type of sensor that could be used to indicate the presence of the person in the space. If the sensors indicate that there is no person in the space then the method 700 can proceed to step 716 where the valve can be opened allowing disinfectant into the proper conduit of the fluid distribution network, and the nozzles for the corresponding group in the space to be disinfected are then activated, wherein the disinfectant is dispersed into the space. However, if in step 710 person seems to be present, the system will indicate a problem in step 712, such as by recording an event fail in a log, as well as potentially transmitting alert signal to an assigned person or personnel. In step 714 a determination can be made whether to abort the disinfection of the particular space. For example, the system via the controller can activate alerting devices in the space, including lights and/or audio alerts to give people in the space time to leave. In which case the method can return to step 710. If, after a number of attempts have been made to clear people from the space, then from step 714 the method can simply end going to step 722, or alternatively to step 728 to determine if there is another space to be disinfected according to the schedule.

In step 716, upon commencing the aerosol disinfection, access controls can be activated, such as a door lock, to exclude people from the space during the disinfection process. Likewise, alerting devices such as a light or lights or sign outside of the space being disinfected can be activated to give people a visual indication that the disinfection process is underway, and that access to the space is currently prohibited. As the disinfection process continues the method 700 proceeds to step 718 in which the system may determine if there is any override signal that has been received. An override signal can be activated by a person in the space who may not have been detected in step 710. Likewise, if a person is subsequently detected in the space being disinfected, such as by using the same sensors used in step 710, the system can initiate an override. When override event occurs the method proceeds from step 718 to step 724 in which disinfection for this particular group is then shut down. Meaning, that the pump is shut down or shut off, the controller will transmit a message to the nozzles of the particular group to deactivate, whereupon each of the nozzle assemblies will close its respective nozzle valve, and the access controls may be released by the system controller to allow egress out of the space to avoid any further exposure to the disinfectant that had been introduced into the space. Returning to step 718, while no override may be detected, the controller also keeps track of the time and in step 726 determines whether the disinfection time. For the particular group, and the particular space being disinfected, has expired. When, in step 726, it is determined that the time has expired for the space presently being disinfected, the method 700 proceeds to step 728 to determine whether, according to the schedule, or the unscheduled disinfection request, there is another space to be disinfected. If no other spaces to be disinfected than the schedule, or the unscheduled disinfection request has been fully processed and the method ends at step 722. If, in step 728, the schedule, or the unscheduled disinfection request indicates another space to be disinfected, then the method 700 can return to step 708 to repeat part of the method 700 for the next space to be disinfected. Thus, the method 700 proceeds to disinfect each space according to the schedule or the unscheduled disinfection request.

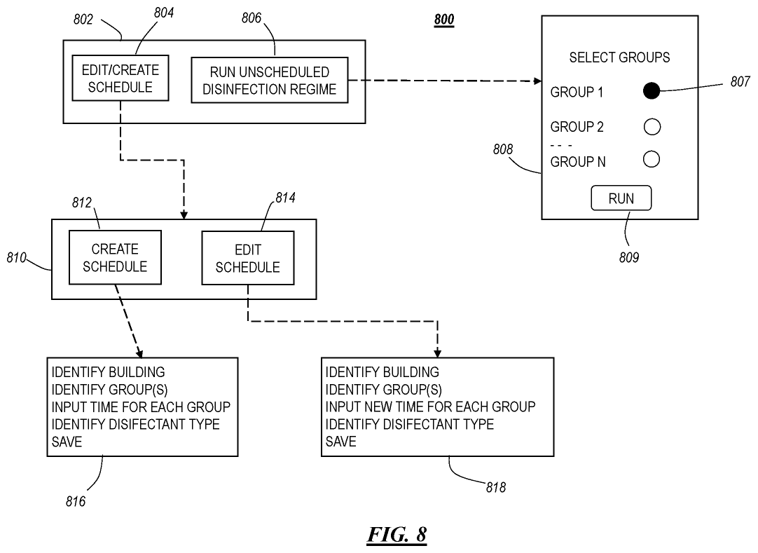

FIG. 8 shows an interface diagram of sequential interface presentations 800 to a user for editing, creating a disinfection schedule or running an unscheduled disinfection operation, in accordance with some embodiments. The interface presentations 800 represent exemplary interfaces or interface elements that can be presented in a graphic user interface of a computing device, including a computer or an application of a smart phone device or similar computing device. In some embodiments the interface can be a web interface presented by a server to a client device accessing the server, or the interface can be presented by an application running locally on a computing device. For example, interface 802 can present to selectable options 804, 806. These selection options can be presented as part of a menu or similar interface hierarchy. Option 804 corresponds to a selection to edit or create a schedule, meaning to edit an existing schedule or create a new schedule. Option. That is, option 806 can be selected to commence an unscheduled disinfection process according to step 706 of FIG. 7. When the user wants to edit or create a schedule the user can select option or interface element 804, such as by pressing on a touchscreen over an interface element corresponding to interface element 804, or using a pointing device (e.g. a mouse) to click on an interface element corresponding to element 804, whereupon a next interface 810 will be brought up giving the user the option to create a schedule in interface element 812, or to edit an existing schedule and interface element 814. If the user selects interface element 812 then a form interface 816 can be displayed to the user in which the user can identify a building various group identifiers and provide a time for each group, and identify a disinfectant type for each group, and then save this information to create a schedule. If, in interface 810, the user selects interface element 814, the user will be displayed a form 818 that already has fields for the schedule populated, which the user can then edit and save. Returning to interface 802, if the user selects interface element 806, then an interface can be displayed allowing the user to select a building and then one or more groups in an interface 808, such as by clicking or selecting a radio button 807 corresponding to a group, and then clicking on a run button 809 to commence the unscheduled disinfection process. It will be realized by those skilled in the art that a near infinite number of various types of interfaces can be designed and displayed in various hierarchies and menus to be displayed to a user.

A disinfection system for spaces in a structure has been disclosed that allows for automatic, scheduled and unscheduled disinfection of spaces through the use of aerosolized disinfectant agents that are distributed to the spaces from a centralized location. The system includes a disinfectant bank that is coupled to a pump. The pump is operable to pump disinfectant material from the disinfectant bank through selected conduits of a fluid distribution network. Each conduit leads to one of more nozzle assemblies connected at the terminal end of the conduit in a space. The system can be operated according to a schedule in which spaces are treated with disinfectant at particular times, and the schedule can be created remotely by accessing a server that maintains schedule information for the system. Upon the time occurring to apply a disinfectant, the system can use one or more sensors to determine whether there are people in the space before commencing the disinfection process, and during the disinfection process the system can control access to the space by locking doors to the space.

What is claimed is:

1. An automated disinfection system, comprising:
a controller;
a pump that is coupled to and operated by the controller;
a reservoir including at least one disinfectant cartridge that contains a disinfectant agent, the reservoir being fluidly coupled to the pump;
a fluid distribution network comprising:
a plurality of conduits, each conduit routed to a different one of a plurality of spaces and separately connected to the pump by a respective one of a plurality of controllable valves at a pump end of each one of the plurality of conduits;
each one of the plurality of conduits being branched, at a terminal end, to a plurality of remotely controlled nozzles, each one of the plurality of nozzles being disposed in one of the plurality of spaces; and
wherein the controller is operable to:
according to a schedule, operate the pump, the plurality of controllable valves, and remotely controlled nozzles to sequentially distribute disinfectant from the disinfectant bank to each one of the plurality of spaces.

2. The automated disinfection system of claim 1, further comprising, in at least one of the plurality of spaces, at least one biosensor that is operably coupled to the controller, wherein the biosensor indicates to the controller whether a person is present in the at least one of the plurality of spaces, and wherein upon detecting a person in the at least one of the plurality of spaces the controller inhibits administering the disinfectant in the at least one of the plurality of spaces in which the person was detected.

3. The automated disinfection system of claim 1, further comprising, in at least one of the plurality of spaces, an access control that is operably coupled to the controller, and which is operated by the controller to prevent access to the at least one of the plurality of spaces upon commencement of administration of the disinfectant into the at least one of the plurality of spaces.

4. The automated disinfection system of claim 1, wherein the plurality of nozzles are each remotely controlled using wireless network communications, and wherein at least of the plurality of nozzles are controlled by the controller as a group with one of the plurality of spaces.

5. The automated disinfection system of claim 1, wherein the controller and pump are housed together on a mobile platform.

6. The automated disinfection system of claim 1, wherein the controller and pump are housed together on a fixed platform in a structure.

7. The automated disinfection system of claim 1, wherein the controller includes a wireless network communication transceiver, and wherein the schedule is received from a remote server via the wireless network communication transceiver.

8. An automated disinfection system, comprising:
a controller including instruction code stored in a non-volatile computer readable medium that is instantiated by the controller for execution by the controller;
a pump that is coupled to and operated by the controller;
a reservoir including at least one disinfectant cartridge that contains a disinfectant agent, the reservoir being fluidly coupled to the pump;
a valve manifold coupled to the pump at an intake and including a plurality of controllable valves;
a fluid distribution network comprising:
a plurality of conduits, each conduit routed to a different one of a plurality of spaces and separately connected to the pump by a respective one of the plurality of controllable valves at a pump end of each one of the plurality of conduits;
each one of the plurality of conduits being branched, at a terminal end, to a plurality of remotely controlled nozzles, each one of the plurality of nozzles being disposed in one of the plurality of spaces;
a plurality of sensors distributed among the plurality of spaces, where each one of the plurality of spaces include at least one sensor, and each sensor of the plurality of sensors being coupled to the controller and operable to indicate to the controller a signal indicating a presence of a person in the respective space; and
wherein the controller is operable to, according to a schedule, operate the pump, the plurality of controllable valves, and remotely controlled nozzles to sequentially distribute disinfectant from the disinfectant bank to each one of the plurality of spaces, and to further inhibit a scheduled disinfection process for a given one of the plurality of spaces when the respective sensor in the given one of the plurality of spaces indicates a person is in the given one of the plurality of spaces.

9. The automated disinfection system of claim 8, wherein the at least one sensor includes a thermal imaging sensor capable of identifying a person, a temperature of the person, and wherein the controller is configured to determine whether the temperature of the person is above an preselected threshold, and to commence an unscheduled disinfection of the space in which the person is located.

10. The automated disinfection system of claim 8, further comprising, in at least one of the plurality of spaces, an access control that is operably coupled to the controller, and which is operated by the controller to prevent access to the at least one of the plurality of spaces upon commencement of administration of the disinfectant into the at least one of the plurality of spaces.

11. The automated disinfection system of claim 8, wherein the plurality of nozzles are each remotely controlled using wireless network communications, and wherein at least of the plurality of nozzles are controlled by the controller as a group with one of the plurality of spaces.

12. The automated disinfection system of claim 8, wherein the controller and pump are housed together on a mobile platform.

13. The automated disinfection system of claim 8, wherein the controller and pump are housed together on a fixed platform in a structure.

14. The automated disinfection system of claim 8, wherein the controller includes a wireless network communication transceiver, and wherein the schedule is received from a remote server via the wireless network communication transceiver.

15. A method for disinfecting a plurality of spaces, comprising:
at a controller, determining that a disinfection process is to commence for at least one of a plurality of spaces;
responsive to determining that the disinfection process is to commence, the controller receiving a signal from a sensor in the at least one of the plurality of spaces;
when the signal indicates that the least one of the plurality of spaces does not contain any persons, the controller:
activating one of a plurality of valves of a valve manifold and opening the valve;
transmitting a signal that causes at least one nozzle in the space to activate;
activating a pump that is coupled to a disinfectant bank on a first side of the pump and to the valve manifold on a second side of the pump to commence pumping disinfectant into the valve manifold;
wherein the disinfectant is pumped through valve manifold into a respective one of a plurality of conduits that is connected at a pump end to the one of the plurality of valves in the valve manifold and at a terminal end to the at least one nozzle; and
the at least one nozzle distributing aerosolized disinfectant into the space.
